(19) United States
(12) Patent Application Publication
(10) Pub. No.: US 2015/0223737 A1
(43) Pub. Date: Aug. 13, 2015

(54) In Vivo Determination of a Blood Value

(71) Applicant: SIEMENS AKTIENGESELLSCHAFT, München (DE)

(72) Inventors: David Groke, Nurnberg (DE); Michael Wiets, Erlangen (DE)

(21) Appl. No.: 14/422,787

(22) PCT Filed: Aug. 27, 2013
(86) PCT No.: PCT/EP2013/067674

§ 371 Date: Feb. 20, 2015

(30) Foreign Application Priority Data
Aug. 30, 2012 (DE) 10 2012 215 389.6

(51) Int. Cl. A61B 5/1459; A61B 5/00; A61B 5/1455

(52) U.S. Cl. CPC A61B 5/1459; A61B 5/14551; A61B 5/0004; A61B 5/742

(57) ABSTRACT

A device is provided for in vivo determination of at least one blood value within an object to be examined. The device includes a catheter-shaped carrier that may be inserted into the object to be examined. The catheter-shaped carrier has at least one measuring device, of which the measurement position is arranged at a definable position of the catheter-shaped carrier, and which is designed to repeatedly determine at least one blood value and to supply this value to an evaluation unit. Moreover, a corresponding method is described for in vivo determination of at least one blood value within an object to be examined.

### CROSS-REFERENCE TO RELATED APPLICATIONS

The present patent document is a §371 nationalization of PCT Application Serial Number PCT/EP2013/067674, filed Aug. 27, 2013, designating the United States, which is hereby incorporated by reference, and this patent document also claims the benefit of DE 10 2012 215 389.6, filed on Aug. 30, 2012, which is also hereby incorporated by reference.

### TECHNICAL FIELD

The present embodiments relate to a device for in vivo determination of at least one blood value. The present embodiments also relate to a corresponding method for in vivo determination of at least one blood value.

### BACKGROUND

In clinical practice, a number of blood values of the human or animal patient may be determined before a medical examination or a medical intervention. In order to determine the blood values, blood may be taken from the patient for the purpose before the examination or intervention so that there is sufficient time for values of relevance to the examination to be determined before the examination starts and it is possible to take any particularities into account. External analysis equipment may be used to determine the blood values. This may be located in a laboratory belonging to the hospital. If relevant blood values are missing, perhaps because it was not possible to take samples in advance, the examination or intervention may be postponed or even canceled. If there is an emergency situation, patients are examined or treated without values being available, with the result that there is a higher risk, for example, of kidney failure. If blood samples are taken during an examination or intervention, the samples may also be sent to the hospital's own laboratory. In a favorable scenario, analysis equipment is available, for example, in a catheter laboratory, allowing certain blood values to be determined. If the samples have to be sent to a laboratory, the diagnosis is delayed until the examining physician has the data, even if the laboratory is in proximity to the catheter laboratory. Diagnosis may not take place until the blood values have been received from the laboratory.

The determination of cardiac output CO is described here by way of example. Cardiac output is determined, for example, by way of the oxygen partial pressure PO₂ that is measured in two different vessels, or by way of the peripheral capillary oxygen saturation SpO₂. This is the most reliable measuring method, but it is not widely used in everyday clinical practice as it is very complex. Other methods are therefore used, such as the determination of cardiac output from output volume and cardiac frequency, referred to as angio, or measured over a temperature progression, referred to as thermo. One particularly important blood value that has to be taken into account during interventions is the creatinine value. Creatinine is an important kidney retention parameter in laboratory medicine. Kidney retention parameters are measurement values determined in medical laboratories, which are used in medicine, specifically nephrology, to assess kidney performance. An increase indicates limited kidney function, referred to as retention. In such cases, iodide may not be injected into the patient as a contrast agent, as it would not be broken down by the kidneys or would only be broken down slowly and therefore in a manner that is damaging to health. Patients frequently have to be treated angiographically in time-critical emergencies without such a laboratory diagnosis, however, even though there is then always the risk of kidney failure. The following blood parameters or blood values may also be mentioned by way of example but not conclusively: erythrocytes, leucocytes (e.g., neutrophil granulocytes, eosinophil granulocytes, lymphocytes and monocytes), thrombocytes, hemoglobin, hematocrit, mean cell volume (MCV), mean corpuscular hemoglobin (MCH), and mean corpuscular hemoglobin concentration (MCHC).

A method known in principle for determining blood values relates to the non-invasive determination of blood values by reflection spectrometry. The publication “Revolution in der Medizintechnik” (Revolution in medical engineering), SCHOTTsolutions, No. 1/2009, pages 42 to 43, proposes equipment that utilizes this measuring principle. A defined white light spectrum is conducted from a manual device by way of glass fibers into a sensor head and radiated into the tissue below the skin on which it is positioned. Different tissue and blood components either absorb the light or it is reflected differently in a substance-specific manner. The procedure is referred to as reflection spectrometry. The reflected light passes by way of a further light conductor into a spectrometer and is broken down into its wavelengths there. The blood values to be tested are concluded from the spectrum analysis and the measurement values are displayed on a display unit.

### SUMMARY AND DESCRIPTION

The scope of the present invention is defined solely by the appended claims and is not affected to any degree by the statements within this summary. The present embodiments may obviate one or more of the drawbacks or limitations in the related art.

The object of the present embodiments is now to specify a device, which allows in vivo determination of blood values within an examination object. It is also the object of the embodiments to describe a corresponding method for in vivo determination of blood values within an examination object.

A device for in vivo determination of at least one blood value within an examination object includes a catheter-shaped carrier that may be inserted into an examination object. The device further includes at least one measuring device, the measuring position of which is arranged at a definable position of the catheter-shaped carrier and which is configured to determine at least one blood value repeatedly and to supply this value to an evaluation unit.

The device therefore allows at least one blood value of an examination object, (for example, a human or animal patient), to be determined or measured in vivo, (in other words, in the living object). The device has a catheter-shaped carrier, on or in which at least one measuring device is arranged. A catheter-shaped carrier has features of a medical catheter or is a medical catheter. A medical catheter refers, in particular, to a tube-type or rod-type device with a length of approx. 0.3 to 1.5 m and a diameter of approx. 1 to 20 mm, which may be inserted into a human or animal body. A medical catheter may also include integrated instruments or instruments that may be inserted by way of operating channels, (for example, micromechanical devices, such as small forceps or grippers), which may be used to perform examining or intervening procedures. A catheter refers to a medical catheter here and in the following. The catheter-shaped carrier and the at least one measuring device, the measuring position of which on or in the catheter-shaped carrier is known, allow measurements of one or more blood values, (e.g., values of the blood composition or the blood gases), to be performed within the examination object, (for example, in a blood vessel). The measurement may be performed once or a number of times and the measurement values obtained in each instance may be transmitted to an evaluation unit, for example, an electronic circuit or a computer. It is indeed conceivable for the at least one measuring device to be arranged in a spatially distributed manner. Thus, for example, the measuring position of a measuring device may be located with a first part of the measuring device at the distal end of the catheter-shaped carrier and a second part of the measuring device may be located in proximity to the proximal end or even outside the catheter-shaped carrier. Optical or electrical measurement signals may be transmitted by an optical or electrical connection from the first to the second part of the measuring device, where they are converted where applicable to blood values or blood measurement values, which are then available to the evaluation unit. The advantage of this embodiment is therefore, inter alia, that the first part of the measuring device for receiving a, for example, optical, measurement signal, may have a smaller structure than the second part of the measuring device, with the aid of which the measurement signal may be converted to a blood measurement value.

The measuring principle of the measuring device may be based on a spectrometric measuring principle.

As described above, the spectrometric, or in particular the reflection spectrometric, measurement of blood values is based on the principle that different tissue and blood components either absorb incident light or reflect it differently in a substance-specific manner. The reflected light component is analyzed using a spectrometer and the desired blood values are determined therefrom. One advantage of this measuring method is that the structure and composition of the blood are not changed by measuring, as would be the case, for example, with testing using a centrifuge. Also, unlike when an analysis is performed in an external laboratory, the measurement values are available within a short time, (for example, within a few seconds), so the measurement values may be obtained during an examination or intervention.

It is favorable if at least part of the measuring device includes a transmitter, which is designed to supply a blood value wirelessly to the evaluation unit.

This feature provides that the measuring device or, in the case of a multi-part measuring device, at least part of the measuring device is provided for the wireless transmission of an obtained or determined blood value. This may be, for example, a transmitter known per se, (such as a wireless local area network or WLAN), according to a standard from the IEEE-802.11 family, or a transmitter as specified in the industrial standard according to IEEE 802.15.1, also referred to as Bluetooth. If the evaluation unit has a corresponding receiver, it may receive the supplied, in other words, transmitted, blood measurement values.

The catheter-shaped carrier particularly advantageously has at least two openings that form at least one cohesive cavity.

A constant throughflow of blood is possible through the at least two openings in the catheter-shaped carrier. It is conceivable, for example, that a first opening is located at the distal end and a second opening is located on the side of the catheter-shaped carrier. If the measuring principle of the measuring device requires direct contact with the blood to be measured, this is provided by this feature.

In one advantageous development of the device, the catheter-shaped carrier has at least one integrated or insertable catheter instrument.

The fact that the catheter-shaped carrier may be fitted with one or more catheter instruments, or that catheter instruments integrated in the catheter-shaped carrier may be used, provides that the device may be used not only to determine blood values but also as a standard medical catheter, as known in clinical practice.

In a further advantageous embodiment, the at least one measuring device is configured to determine at least one blood value at predefinable time points and to supply this value to the evaluation unit.

By measuring blood values repeatedly, it is possible, (for example, during an intervention), to monitor the progression of a blood value over time and thus identify situations that are dangerous for the patient in a timely manner. The time points or the rate at which measurements are performed may be predefined, for example, by an operator (e.g., a physician).

In a further advantageous embodiment of the device, the evaluation unit is configured to represent the supplied blood value on an output device and/or supply it to a monitoring system and/or supply it to a network and/or record it in a standardized data record.

The device is configured to transmit measured blood values to the evaluation unit, for example, an electronic circuit or a computer. The evaluation unit may also be configured to represent the blood value(s) on an output device, for example, on a computer monitor. This is of particularly significant advantage during a medical intervention, as a physician, for example, may then monitor the current blood value in a live manner and take actions as required. The evaluation unit may also be configured to supply the supplied blood value to a monitoring system. Monitoring may refer to the direct systematic acquisition, in other words the logging, observing or watching, of a procedure or process using technical aids or other observation systems. One important aspect here is the repeated regular performance of test programs. It is also conceivable to intervene in a regulating manner in an observed procedure or process, if it does not follow a desired progression or if results are below or above predefinable limit values. One commercially available monitoring system is known, for example, under the name AXIOM Sensis XP and is produced by Siemens. The evaluation unit may also be configured to supply the blood value(s) to a network, by feeding the data into a data processing network by way of suitable interfaces in a wired or wireless manner. The determined blood value provided by the evaluation unit may be recorded in a standardized data record, (e.g., in a DICOM Structured Report), for further diagnosis.

In a further embodiment, a method for in vivo determination of blood values is provided. The method includes the following acts. In act S1, at least one blood value is determined using at least one measuring device, the measuring position of which is arranged at a definable position of a catheter-shaped carrier and which is inserted into an examination object. In act S2, the at least one blood value is supplied to an evaluation unit.

In one embodiment of the method, at least one time point may advantageously be predefined, at which the measurement of the at least one blood value is performed.

In a further advantageous embodiment, the method is used with one of the devices described above for in vivo determination of at least one blood value within an examination object.

When one of the devices described above is used, method acts for which the embodiment of the respective device is designed are particularly advantageously executed.

In a further advantageous embodiment of the method, the method is executed automatically.

Automatically executed methods have the advantage that an operator is required to perform fewer interventions, which are often time-consuming and error-prone.

### BRIEF DESCRIPTION OF THE DRAWINGS

FIG. 1 depicts a schematic diagram of an exemplary embodiment of a device for in vivo determination of a blood value within an examination object.

FIG. 2 depicts a schematic diagram of an exemplary embodiment of a device for in vivo determination of a blood value within an examination object with a standard catheter function.

FIG. 3 depicts an example of a flow diagram of a method for in vivo determination of a blood value within an examination object.

FIG. 4 depicts a schematic diagram of an exemplary embodiment of a device for in vivo determination of a blood value within an examination object, the device being incorporated in a medical system.

### DETAILED DESCRIPTION

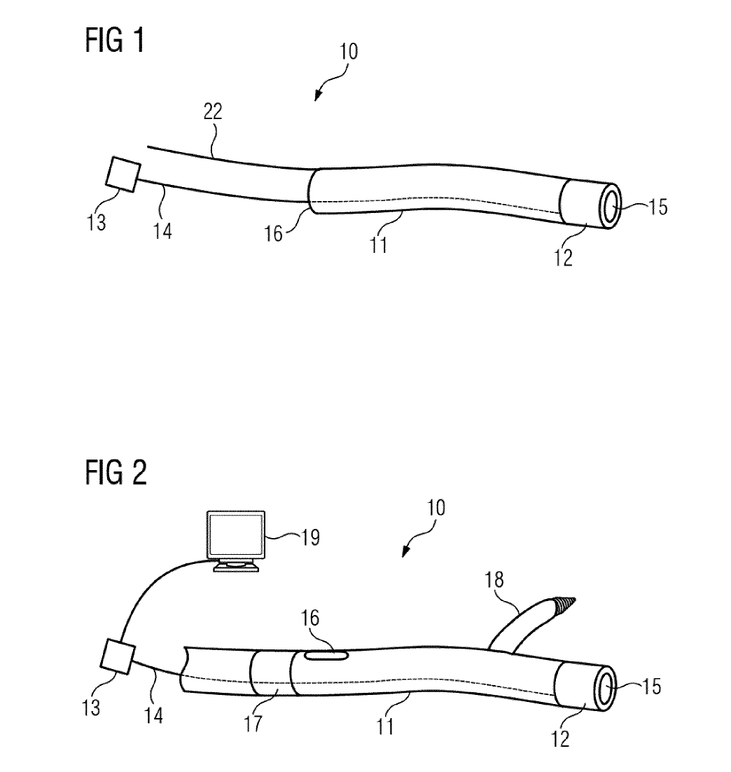

FIG. 1 depicts a schematic diagram of an exemplary embodiment of a device 10 for in vivo determination of a blood value within an examination object. The device 10 has a catheter-shaped carrier 11, on which a measuring device 12 is arranged. The catheter-shaped carrier 11 in this exemplary embodiment is a tube-type device, which may be inserted into a human or animal body. The catheter-shaped carrier 11 has two openings 15 and 16 at its ends, forming a cohesive cavity. This allows, for example, an almost unimpeded blood flow through the tube-type carrier 11. A guide wire 22 serves to navigate the catheter-shaped carrier 11. The measuring principle of the measuring device 12 is based on a spectrometric measuring principle, which may be used to determine, for example, values of the blood composition or the blood gases. The measuring position of the measuring device 12 on the catheter-shaped carrier 11 is known. In this exemplary embodiment, the measuring device 12 is arranged at the distal end of the carrier 11 so that when the location of the carrier 11 within the examination object is known, the measurement site of an obtained measurement value is also known. A measurement value may be transmitted to an evaluation unit 13, (e.g., an electronic circuit or a computer), by a connector 14, e.g. a metal cable.

FIG. 2 depicts a schematic diagram of an exemplary embodiment of a device 10 for in vivo determination of a blood value within an examination object, (e.g., a human or animal patient), with a standard catheter function. In this exemplary embodiment, the device 10 has a catheter-shaped carrier 11, on which two measuring devices 12 and 17 are arranged with defined measuring positions. It is possible by the catheter-shaped carrier 11 and the two measuring devices 12 and 17 to perform measurements of one or more blood values, (e.g., values of the blood composition or the blood gases), within the examination object, (for example, in a blood vessel). Two openings 15 and 16 in the catheter-shaped carrier 11, the first opening 15 being located at the distal end and the second opening 16 being located on the side of the catheter-shaped carrier 11, allow a constant throughflow of blood, even if the proximal end of the catheter-shaped carrier 11 is located outside the examination object. A measurement may be executed once or a number of times, for example, at a constant measuring rate. The measurement values are transmitted to an evaluation unit 13, (e.g., an electronic circuit or a computer), by a connector 14, (e.g., a metal cable). Wireless transmission of the measurement values from both measuring devices 12 and 17 to the evaluation unit 13 is also conceivable, it being possible to consider the connector 14 to be a wireless data transmission link. An output device 19, (for example, a computer monitor), represents the determined and transmitted measurement values visually. A catheter instrument 18, in this instance a micromechanical drill, which may be used to perform examining or intervening procedures, is also integrated in the catheter-shaped carrier 11. Other catheter instruments of a medical catheter known per se, such as small forceps or grippers, which may also be embodied as instruments that may be inserted into the catheter-shaped carrier 11, are also conceivable. The fact that catheter instruments may be used provides that functions of a standard catheter may also be performed with the device 10.

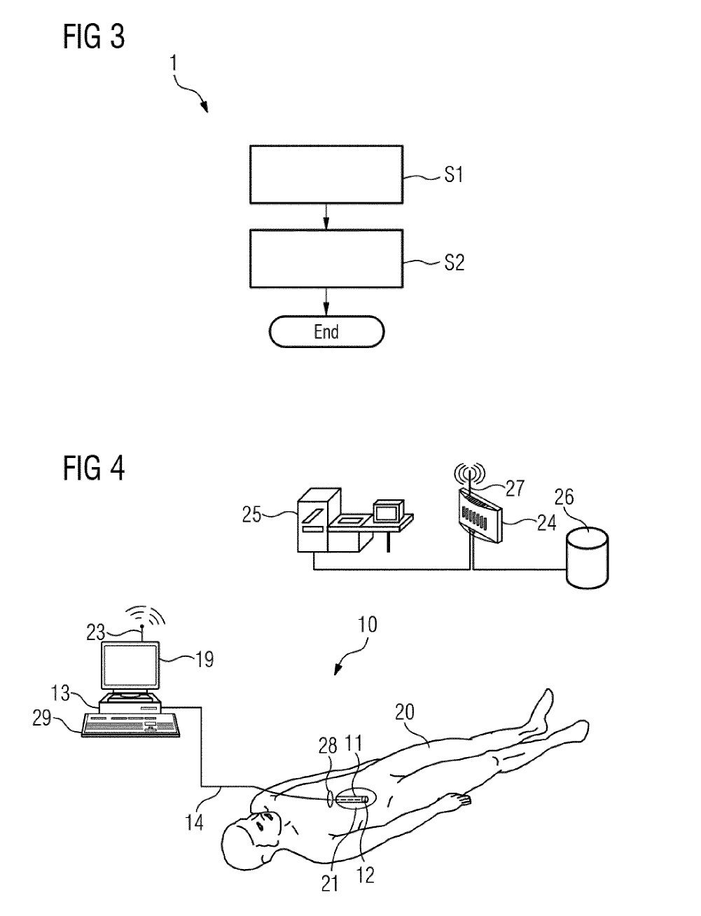

FIG. 3 depicts an example of a flow diagram of a method 1 for in vivo determination of at least one blood value within an examination object. The method 1 includes acts S1 and S2. It starts with act S1 and ends, “End”, after act S2. The individual acts are as follows. In act S1, at least one blood value is determined using at least one measuring device, the measuring position of which is arranged at a definable position of a catheter-shaped carrier and which is inserted into an examination object. In act S2, the at least one blood value is supplied to an evaluation unit.

Finally, FIG. 4 depicts a schematic diagram of an exemplary embodiment of a device 10 for in vivo determination of a blood value within an examination object 20, in this instance a human patient, the device 10 being incorporated in a medical system. The device 10 includes a catheter-shaped carrier 11, which may be inserted through a body entry opening 28 of the examination object 20 into an examination region 21, (for example, a vein, such as the vena subclavian). A measuring device 12, the measuring position of which is depicted schematically at the distal end of the catheter-shaped carrier 11, allows the measurement of blood values, (for example, the creatinine value), locally in the examination region 21 using a spectrometric measuring principle. Measurement values from the measuring device 12 are transmitted to an evaluation unit 13, in this instance a computer, by way of a connector 14, for example, a data cable. The evaluation unit 13 may represent the blood value(s) on an output device 19, in this instance a computer monitor. This is of significant advantage, for example, during a medical intervention, as a physician may then monitor the current blood value in a live manner and take actions as required. The measurement, in particular, the selection of the type of blood value to be measured and the time points at which measurement values are taken, may be controlled by an operator, (e.g., the treating physician), for example, by inputting at an input device 29, (in this instance, a computer keyboard). In this exemplary embodiment, the evaluation unit 13 is further configured to supply the supplied blood value to a monitoring system 25. This is done by wireless data transmission using a transmitter 23, depicted here symbolically by a transmit antenna, and a receiver 27, in this instance, a receive device with receive antenna. The monitoring system 25 is used, inter alia, for the systematic acquisition and where applicable representation of the measurement values. The determined blood value(s) supplied by the evaluation unit is/are also included in a standardized data record 26, (for example, a DICOM Structured Report), and are available there for further diagnosis.

The evaluation unit 13 is configured to supply the blood value(s) to a network 24 by feeding the data into a data processing network by way of suitable interfaces in a wired or wireless manner. In this exemplary embodiment, the network 24 is depicted symbolically by a receive device. In one embodiment, the network 24 may be a networked hospital information system made up, for example, of a number of decentralized computers that may communicate with one another in a wireless or wired manner.

In conclusion, some features of exemplary embodiments and advantages are summarized. With the aid of a device, (for example, a catheter), which is designed to execute the described functions, a user may save on time and personnel outlay, the time factor may be more significant. There is also constant monitoring and representation of the blood values, for example, the oxygen saturation in the blood. An examination and/or intervention may now be performed on patients for whom there is no current blood picture and therefore no examination and/or intervention may be performed under current guidelines. It may also be documented simultaneously and automatically the blood values that have changed during the examination and/or intervention, at what time and under which circumstances. Conclusions may also be drawn as to the medication the patient requires. One significant advantage is the possibility of representing the measured blood values online. The examining physician sees the body's reaction to the examination method instantly. As described above the creatinine value in particular rises when x-ray contrast agent containing iodide is administered. When a critical tolerance value is reached, the examining physician may terminate the examination in a timely manner or inject fluid, (for example, a NaCl solution), before the patient's kidneys are damaged.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A device for in vivo determination of at least one blood value within an examination object, the device comprising:
a catheter-shaped carrier configured to be inserted into the examination object with at least one measuring device,
wherein a measuring position of the measuring device is arranged at a definable position of the catheter-shaped carrier,
wherein the measuring device is configured to determine the at least one blood value repeatedly and to supply the at least one blood value to an evaluation unit, and
wherein the catheter-shaped carrier comprises at least two openings that form at least one cohesive cavity.

2. The device as claimed in claim 1, wherein a measuring principle of the at least one measuring device is based on a spectrometric measuring principle.

3. The device as claimed in claim 1, wherein the measuring device comprises a transmitter configured to supply a blood value wirelessly to the evaluation unit.

4. (canceled)

5. The device as claimed in claim 1, wherein the catheter-shaped carrier comprises at least one integrated or insertable catheter instrument.

6. The device as claimed in claim 1, wherein the at least one measuring device is configured to determine the at least one blood value at predefinable time points.

7. The device as claimed in claim 1, wherein the evaluation unit is configured for one or more of the following: (1) to represent the blood value on an output device, (2) to supply the blood value to a monitoring system, (3) to supply the blood value to a network, or (4) to record the blood value in a standardized data record.

8. A method for in vivo determination of blood values within an examination object, the method comprising:
determining at least one blood value using at least one measuring device, a measuring position of which is arranged at a definable position of a catheter-shaped carrier inserted into the examination object;
supplying the at least one blood value to an evaluation unit.

9. The method as claimed in claim 8, wherein at least one time point is predefined, at which the measurement of the at least one blood value is performed.

10. The method as claimed in claim 9, wherein the in vivo determination of at least one blood value within the examination object is performed by a device comprising a catheter-shaped carrier having at least two openings that form one cohesive cavity

11. The method as claimed in claim 10, wherein the method is executed automatically.

12. The method as claimed in claim 8, wherein the in vivo determination of at least one blood value within the examination object is performed by a device comprising a catheter-shaped carrier having at least two openings that form one cohesive cavity.

13. The method as claimed in claim 12, wherein the method is executed automatically.

14. The method as claimed in claim 8, wherein the method is executed automatically.

15. The device as claimed in claim 2, wherein the measuring device comprises a transmitter configured to supply a blood value wirelessly to the evaluation unit.

16. The device as claimed in claim 15, wherein the catheter-shaped carrier comprises at least one integrated or insertable catheter instrument.

17. The device as claimed in claim 16, wherein the evaluation unit is configured for one or more of the following: (1) to represent the blood value on an output device, (2) to supply the blood value to a monitoring system, (3) to supply the blood value to a network, or (4) to record the blood value in a standardized data record.

18. The device as claimed in claim 3, wherein the catheter-shaped carrier comprises at least one integrated or insertable catheter instrument.

19. The device as claimed in claim 18, wherein the evaluation unit is configured for one or more of the following: (1) to represent the blood value on an output device, (2) to supply the blood value to a monitoring system, (3) to supply the blood value to a network, or (4) to record the blood value in a standardized data record.

20. The device as claimed in claim 3, wherein the evaluation unit is configured for one or more of the following: (1) to represent the blood value on an output device, (2) to supply the blood value to a monitoring system, (3) to supply the blood value to a network, or (4) to record the blood value in a standardized data record.
